(19) United States
(12) Patent Application Publication
(10) Pub. No.: US 2006/0167392 A1
(43) Pub. Date: Jul. 27, 2006

(54) Adjustable leg sling

(76) Inventor: Jennifer J. Manquez, Siloam Springs, AR (US)

Correspondence Address: Raymond M. Galasso;Galasso & Associates LP, P.O. Box 26503, Austin, TX, 78755-0503, US

(21) Appl. No.: 11/337,281

(22) Filed: Jan. 23, 2006

Related U.S. Application Data
(60) Provisional application No. 60/646,289, filed on Jan. 24, 2005.

(51) Int. Cl. A61F 5/00

(52) U.S. Cl. 602/4; 602/23

(57) ABSTRACT

A low cost, compact, lightweight sling is disclosed. It can be appreciated that moving an impaired leg often requires someone to bend down and place their hands near the ground to grasp the leg. For many, this requirement to bend over is burdensome and can leave on in an awkward situation. In accordance with the present invention, an impaired leg can be moved without the requirement of bending over to grasp the leg, then lift the leg. The sling can have a pull strap, a boot and a coupler to removably affix the pull strap to the boot. The sling can be manufactured with a strap, such as a nylon strap that has a loop on a first end of the strap that can secure a handle. On a second end of the pull strap a foot engaging/securing member such as a boot can be configured to secure a human foot or a shoe. Securing straps can secure the boot to the user's foot. The boot can also be configured with a molded heel to secure the users foot and prevent the boot from slipping off of the users foot. In one embodiment the boot can have reinforcing straps that are affixed to the boot such that the pull strap can be secured to the boot.

### PRIORITY UNDER 35 U.S. C. §619(e) & 37 C.F.R. §1.78

This nonprovisional application claims priority based upon the following prior United States provisional patent application entitled: Leg Sling, Application No. 60/646,289, filed Jan. 24, 2005, in the name of Jennifer J. Marquez, which is hereby incorporated by reference for all purposes.

### FIELD OF THE DISCLOSURE

The present disclosure is generally related to systems and methods for controlling, and assisting in, the movement of an impaired human's limb and more specifically to methods and arrangements for assisting a human in the movement of their leg.

### BACKGROUND

Many individuals become injured through car accidents, work related accidents, and participating in sports. Phenomena such as strokes, spinal cord injury, and brain damage can also affect one's ability to move their legs. Many events can occur that create some form of paralysis in the leg or some inability to fully move the leg. When an injured individual cannot lift their legs they often require assistance from others. For example, simple tasks like getting in and out of a car, a bed, or a bath tub can be a significant challenge for one who has limited mobility in their legs. Further, someone who is assisting a person with limited leg mobility often must bend over and grab the leg of the impaired and lift the leg to place it in the desired position. This lifting procedure most always occurs from an awkward position such as when impaired person is trying to exit a car. In this case the person assisting has to lean over and duck into the car, reach to the floor of the car and lift the impaired person's let out of the car. First, an assistant has to be mobile, and second this can cause injury to the assistant. Accordingly, it would be desirable to have an improved system and method for allowing an injured individual to move his legs and to allow for a person assisting the impaired individual to easily assist the injured individual. Further, medical doctors often prescribe physical therapy for the injured individual. It is well known that when an impaired individual is ready, therapy can assist in a recovery process. However, not all impaired individuals have access to expensive therapy equipment. Additionally, cost effective leg therapy device would be desirable.

### BRIEF DESCRIPTION OF THE DRAWINGS

FIG. 1 is an illustration of a leg sling in accordance with one embodiment of the present disclosure.

### DETAILED DESCRIPTION OF THE DRAWINGS

In accordance with the present invention, a device for assisting or resisting the movement of a human leg is provided. Many individuals have limited use of their legs and need assistance in moving their legs, while others who need to build strength in their legs can require gentle forms of therapy. For many injured and elderly it is difficult to accomplish simple tasks such as getting into an out of a car because they cannot lift their leg high enough or they cannot lift their legs at all. Further, for those who cannot lift their legs on their own or have limited mobility, tasks such as getting into or out of a bath tub or a bed can be very difficult particularly when left unsupervised.

In one embodiment of the present invention a foot securing member or boot is provided that can be secured to the foot area of an impaired individual. A pull strap with a handle can be connected to the boot. The pull strap can be pulled on to move the leg of the impaired or could be utilized for the dual purpose of supporting a leg while the impaired person lies in a bed. In a specific embodiment, if the impaired individual has use of their arms, the impaired individual can pull on the pull strap to move their legs without requiring the help of others. In another embodiment, someone who is assisting the impaired individual can pull on the pull strap and guide the legs of the impaired individual into a desired position.

It can be appreciated that moving an impaired leg often requires someone, possibly the individual with the impaired leg, to bend down and place their hands near the ground to grasp the leg or foot and then lift the leg. For many, this requirement to bend over and lift awkward and burdensome. In accordance with the present invention, an impaired leg can be moved without the requirement of bending over to grasp the leg or foot.

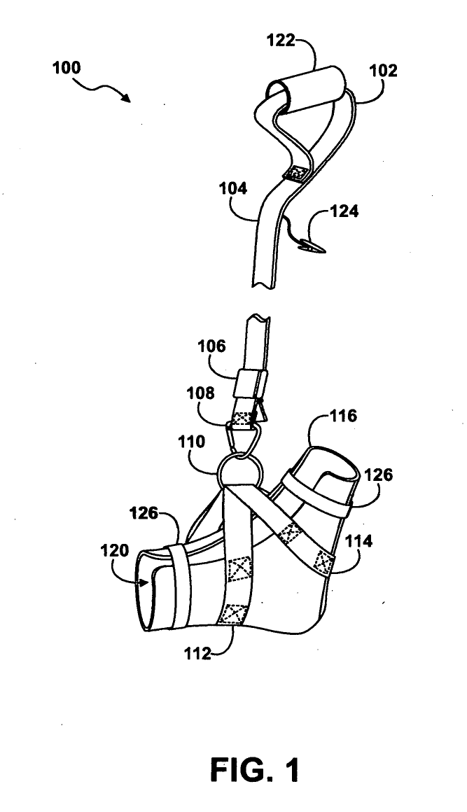

Referring to FIG. 1, a low cost, compact, lightweight sling 100 is illustrated. The sling 100 can be manufactured with a pull strap 104, such as a nylon strap or a wire rope. The pull strap 104 can have a loop 102 on a first end that can secure a handle 122. The loop 102 can be formed by folding the pull strap 104 over on to itself threading the pull strap 104 through a tubular handle 122 and sewing the end of the pull strap 104 to a location on the pull strap 104. The size of the loop 102 and the shape of the handle 122 should be configured such that a human can obtain a firm grip on the handle 122 or the pull strap 104. In a specific embodiment, the pull strap 104 can have a buckle 106 other means for adjusting the length of the strap. Generally, the length of the pull strap 106 can be adjustable from 24 to 60 inches. Thus the length of the pull strap can be adjusted depending on the size of the impaired individual or depending on who is required to utilize the pull strap 106. The pull strap 104 could be embodied as a single piece or material or multiple segments of material.

On a second end of the pull strap 104, a foot engaging/securing member such as a boot 116 can be configured to secure a foot or a shoe of the impaired individual. The boot 116 can be made from a fabric type material or it can be made from a semi-rigid material such as plastic or rubber. The boot 116 can also have a rigid sole with fabric flaps or could be made from molded plastic or rubber. The boot 116 could also be a substantially flat member until it is wrapped around the shoe or foot of the user and the boot 116 could have a cut out for the toes and the heel.

The boot 116 can have fastener straps 126 that can wrap around the wearer's foot thereby securing the boot 116 to the user's foot 126. Fastener straps 126 could use hook and loop fasteners to secure the boot 116 to the sides of the wearer's foot. The hook fasteners can be connected to a first end of the fastener straps 126 on one side of the boot 116 and the loop fasteners can be connected to a second end of the fastener straps 126 on another side of the boot 116 such that when the boot 116 is wrapper around a foot, the fastener straps 126 can secure the user's foot. In other embodiments the boot 116 can just cradle the wearer's foot.

Hook and loop fasteners are often referred to by a trade name “Velcro™”. Fastening mechanisms other than Velcro could also be utilized at the ends of the fastener straps 126 without parting from the scope of the disclosure. For example, a buckle, shoe laces, an over the center latch or other mechanism could be utilized to secure fastener straps 126 around a foot or shoe.

The boot 116 can also be configured as tubular, stretchy “sock” a plastic member with a foot and heel support or just a series of straps. A main goal of the boot 116 is to secure the users foot and prevent the boot 116 from slipping off of the users foot at inopportune times.

In one embodiment the boot 116 can have reinforcing straps 112 and 114 that are affixed to the boot 116 by some fastening method such as gluing or sewing. Reinforcing straps 112 and 114 can create a loop on the top side of the boot 116 such that a ring 110 and pull strap 104 can be connected to the boot 116.

A coupler 108 could be utilized to secure a second loop on the second end of the strap 104 to the ring 110. Thus the strap 104 could be removably coupled to the boot 116. In one embodiment, after assisting in moving the legs of an impaired individual, the strap 104 can be disconnected from the boot 116 such that the strap 104 does not interfere with other activities. Coupler 108 could be an aluminum carabineer that a spring loaded locking member wherein the coupler 108 cannot become disconnected from the ring 110 without an individual unlocking a clasping member of the coupler 108.

In a specific embodiment a clip 124 can be sewn to the strap 104 such that when the sling 100 is not in use, the loop end of the sling 100 can be fastened onto a piece of clothing or a belt worn by the individual with the impaired leg. This allows anyone including the impaired individual to easily locate the handle 122 or loop 102. Otherwise it is possible for the handle 122 to fall on the floor and get dirty, lost or tangled. In another embodiment, instead of a clip 124, Velcro, a hook or another fastening means could also be utilized.

In one embodiment a physical therapist can utilize the sling to provide physical therapy to the impaired individual. Such therapy is often referred to as manual manipulation. A therapist can move the impaired leg by pulling on the strap 102 and in turn the leg that is secured by the boot 116 can receive movement or resistance to movement.

If an individual with an impaired leg has some motor skills in the leg then the sling 100 can also be utilized resist movement of the leg in all directions, thereby assisting the impaired individual in increasing leg mobility and strength. The physical therapist and the sling 100 can reduce the effect of gravity and allow for a user induced movement without the harsh effects of gravity. Such assistance can reduce the danger of further injury due to uncontrolled movement or overloading the muscles, bones and ligaments.

The sling 100 could provide a triple role as a device to raise the level of the leg when a person is lying in bed.

It will be understood that a device as specified by the present disclosure may also be directed to other application of similar functionality. The above-disclosed subject matter is to be considered illustrative, and not restrictive, and the appended claims are intended to cover all such modifications, enhancements, and other embodiments, which fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

What is claimed is:

1. A sling comprising:
a boot comprising;
a sole configured to support a foot;
a reinforcement strap connected to the sole and configured to secure a coupler to the boot; and
a fastening strap secured to the sole and configured to alter a size of the boot;
a pull strap having a first end forming a loop;
a coupler removably couplable to the pull strap and the reinforcing strap; and
a buckle affixed to the pull strap and configured to adjust a length of the pull strap.

2. The sling of claim 1 wherein the pull strap has multiple segments.

3. The sling of claim 1 further comprising a detachable coupler configured to connect the pull strap to the reinforcement strap.

4. The sling of claim 3 wherein the detachable coupler further comprises a latching device.

5. The sling of claim 1 further comprising a clip coupled to the pull strap and configured to fasten the pull strap to a clothing article when the sling is not in use.

6. The sling of claim 1 further comprising hook and loop fasteners on the fastening straps wherein the boot is adjustable in size utilizing the hook and loop fasteners.

7. The sling of claim 1 wherein the loop is formed by folding the pull strap over and sewing the pull strap.

8. The sling of claim 1 further wherein the coupler further comprises a ring configured to connect the pull strap to the boot.

9. A leg sling comprising:
at least one pull strap forming a loop, the loop configured to be held by a hand;
a flexible boot having a first flap connectable with a second flap and a connection point; and
a reinforcing member coupled to the at flexible boot and configured to attach the pull strap.

10. The leg sling of claim 9 further comprising a fastening member coupled to the pull strap and configured to attach the pull strap to an article of clothing worn by a user of the leg sling.

11. The leg sling of claim 10 wherein the fastening member is a clip.

12. The leg sling of claim 9 wherein the flexible boot is adjustable to a size of a human foot.

13. The leg sling of claim 9 wherein the flexible boot is adjustable to a size of a human shoe.

14. The leg sling of claim 10 further comprising a tubular handle wherein the loop traverses the handle.

15. The leg sling of claim 10 further comprising a plurality of securing straps coupled to the boot and configured to wrap around a users foot and secure the users foot in the boot.

16. The leg sling of claim 15 further comprising a buckle configured to assist in adjusting a length of the securing straps.

17. A method comprising:
sewing a strap to form a loop on the strap, the loop configured to be gripped by human hands;
sewing a reinforcing material on a boot configured to cradle a human foot;
coupling the sewn strap to the boot such that pulling on the strap in a direction causes the human foot to move in the pulled direction.

18. The method of claim 17 further comprising sewing hook and loop fasteners on the boot.

19. The method of claim 18 further comprising wrapping the human foot with the boot and securing the boot on the human foot with the hook and loop fasteners.

20. The method of claim 17 further comprising folding the sling into a package measuring less than 7 inches by two inches by two inches.
